(19) United States
(12) Patent Application Publication
(10) Pub. No.: US 2024/0331285 A1
(43) Pub. Date: Oct. 3, 2024

(54) VESSEL PHYSIOLOGY GENERATION FROM ANGIO-IVUS CO-REGISTRATION

(71) Applicant: Boston Scientific Scimed Inc., Maple Grove, MN (US)

(72) Inventors: Kevin Bloms, Minneapolis, MN (US); Wenguang Li, Los Gatos, CA (US)

(73) Assignee: Boston Scientific Scimed Inc., Maple Grove, MN (US)

(21) Appl. No.: 18/622,357

(22) Filed: Mar. 29, 2024

Related U.S. Application Data
(60) Provisional application No. 63/456,335, filed on Mar. 31, 2023.

(51) Int. Cl. G06T 17/00; G06T 7/00

(52) U.S. Cl. CPC G06T 17/00; G06T 7/0012; G06T 2200/24; G06T 2207/10121; G06T 2207/20081; G06T 2207/30104; G06T 2210/24; G06T 2210/41

(57) ABSTRACT

The present disclosure provides apparatus and methods to generate a three-dimensional (3D) model of the physiology of a vessel from a single angiographic image and a series of intravascular images as well as another physical characteristic of the vessel, such as, for example, pressure.

### CROSS-REFERENCE TO RELATED APPLICATIONS

This application claims the benefit of U.S. Provisional Patent Application Ser. No. 63/456,335 filed on Mar. 31, 2023, the disclosure of which is incorporated herein by reference.

### TECHNICAL FIELD

The present disclosure pertains to generating a virtual physiology of a vessel.

### BACKGROUND

A currently accepted technique for assessing the severity of a stenosis in a blood vessel, including ischemia causing lesions, is intravascular imaging combined with angiographic imaging. Further, a physician may use fractional flow reserve (FFR) to assess the stenosis. FFR is a calculation of the ratio of a distal pressure measurement (taken on the distal side of the stenosis) relative to a proximal pressure measurement (taken on the proximal side of the stenosis). FFR provides an index of stenosis severity that allows determination as to whether the blockage limits blood flow within the vessel to an extent that treatment is required. The normal value of FFR in a healthy vessel is 1.00, while values less than about 0.80 are generally deemed significant and require treatment. Common treatment options for stenosis include percutaneous coronary intervention (PCI or angioplasty), stenting, or coronary artery bypass graft (CABG) surgery. As with all medical procedures, certain risks are associated with PCI, stenting, and CABG procedures. For a surgeon to make a better-informed decision regarding treatment options, additional information about the risk and likelihood of success associated with the treatment options is needed.

However, the locations of stenoses in a vessel can be difficult to visualize in a black and white angiographic and IVUS images.

Accordingly, there remains a need for improved devices, systems, and methods for assessing the severity of a blockage in a vessel and a stenosis in a blood vessel. In that regard, there remains a need for improved devices, systems, and methods for providing visual depictions of vessels that allow assessment of the vessel and any stenosis or lesion of the vessel. Further, there remains a need for improved devices, systems, and methods of objectively evaluating risk associated with and likelihood of success for one or more available treatment options for the vessel.

### BRIEF SUMMARY

The present disclosure provides to generate a physiological flow model of a vessel of patient based on a co-registration of a single angiographic image and a series of intravascular images and optionally one or more additional physiological measurement is provided. As a specific example, the present disclosure provides to generate a three-dimensional (3D) model representing at least a portion of the patient's heart based on a series of intravascular ultrasound (IVUS) images co-registered with a single angiographic image.

Accordingly, the present disclosure provides a system to generate a 3D reconstruction of a vessel for providing a virtual physiology of the vessel to a physician. In particular, the present disclosure can be integrated with an intravascular ultrasound assessment system and can avoid using guidewire based assessment systems solely for the purpose of assessing the vessel physiology. Thereby providing an increase in efficiency for the physician and a reduction in the number of invasive procedures with which the patient is subjected to during the assessment of the vessel.

It is to be appreciated that the present disclosure provides a significant advantage over conventional techniques to generate models of vessel physiology. For example, some conventional approaches use multiple angiographic images to generate a vessel physiology model. However, as noted, this approach requires multiple (e.g., two (2) or more) angiographic projections, adding workflow overhead to the procedure. Additionally, border tracing on angiographic images often requires manual correction, further increasing the workflow overhead. Another approach uses a series of IVUS images alone. However, this approach fails to properly account for vessel curvature and side branch sizes, which is necessary for accurate FFR calculations and vessel physiology modeling.

The present disclosure provides to use a single angiographic image and a single series of IVUS images to generate a model of a vessel physiology. This approach corrects inaccuracies in prior approaches, such as, for example, foreshortening and inaccurate vessel dimensioning resulting when a single imaging modality is used to generate the physiology model. The approach of the present disclosure utilized IVUS images to provide accurate distance measurement to correct foreshortening that often results from relying on a single angiographic view to model a vessel. Further, the present disclosure utilizes IVUS cross-sectional area measurements to provides more accurate lumen/vessel dimension than angiographic projections alone can provide. Additionally, the present disclosure utilizes co-registration information between the single angiographic image and the series of IVUS images to provide cross-detection of side branch locations and sizing, thereby providing a more accurate vessel physiology.

In some embodiments, the disclosure can be implemented as a method for generating a 3D model of a physiology of a vessel. The method can include receiving, at a computing device from a fluoroscope device, an angiographic image if a vessel of a patient; receiving, at the computing device from an intravascular imaging device, a plurality of images associated with the vessel of the patient, the plurality of images comprising multidimensional and multivariate images; and generating a three-dimensional (3D) model of a physiology of the vessel from the angiographic image and the plurality of images.

In further embodiments, the method can include generating, by the computing device, a graphical information element comprising an indication of the 3D model; and causing, by the computing device, the graphical information element to be displayed on a display coupled to the computing device.

In further embodiments of the method, generating the 3D model of the physiology of the vessel comprises co-registering the angiographic image and the plurality of images.

In further embodiments, the method can include identifying a start point of a pull-back operation associated with the plurality of images on the vessel represented in the angiographic image; identifying an end point of the pull-back operation associated with the plurality of images on the vessel represented in the angiographic image; and identifying a centerline of the vessel between start point and the end point.

In further embodiments, the method can include identifying a plurality of side branches of the vessel on the angiographic image and in the plurality of images; and matching a one of the plurality of side branches identified on the angiographic image with a one of the plurality of side branches identified in the plurality of images.

In further embodiments, the method can include mapping frames of the plurality of images with locations along the centerline of the vessel on the angiographic image.

In further embodiments, the method can include generating assessments of the vessel.

In further embodiments of the method, the assessments comprise a diameter of the vessel, an area of the vessel, or a diameter and area of the vessel.

In further embodiments of the method, the assessments comprise a diameter of the lumen, an area of the lumen, or a diameter and area of the lumen.

In further embodiments, the method can include receiving, at the computing device, an indication of an additional physiological characteristic of the vessel of the patient; and generating the 3D model of the physiology of the vessel from the angiographic image, the plurality of images, and the additional physiological characteristic of the vessel.

In further embodiments of the method, the additional physiological characteristic of the vessel comprises pressure or flow.

In further embodiments, the method can include generating an inference of the 3D model of the physiologic of the vessel from a machine learning (ML) model based in part on applying the angiographic image and the plurality of images as inputs to the ML model.

In further embodiments of the method, the ML model is trained based in part on a supervised learning training algorithm with expected outputs of the ML model derived based on a computation fluid dynamics (CFD) model, wherein the CFD model takes an angiographic image and a plurality of images as input and generates a 3D vessel physiology model as output.

With some embodiments, the disclosure can be implemented as an apparatus comprising a processor arranged to be coupled to an intravascular imaging device and a fluoroscope device, the apparatus further comprising a memory comprising instructions, the processor arranged to execute the instructions to implement the method of any of the embodiments described herein.

In some embodiments, the disclosure can be implemented as a computer-readable storage device, comprising instructions executable by a processor of a computing device coupled to an intravascular imaging device and a fluoroscope device, wherein when executed the instructions cause the computing device to implement the method of any of the embodiments described herein.

With some embodiments, the disclosure can be implemented as an apparatus for a vascular imaging medical device. The apparatus can include a processor arranged to be coupled to an intravascular imaging device and a fluoroscope device; and a memory device coupled to the processor, the memory device comprising instructions, which when executed by the processor cause the apparatus to: receive, from the fluoroscope device, an angiographic image if a vessel of a patient; receive, from the intravascular imaging device, a plurality of images associated with the vessel of the patient, the plurality of images comprising multidimensional and multivariate images; and generate a three-dimensional (3D) model of a physiology of the vessel from the angiographic image and the plurality of images.

With further embodiments of the apparatus, the instructions when executed by the processor further cause the apparatus to generate a graphical information element comprising an indication of the 3D model; and cause the graphical information element to be displayed on a display coupled to the computing device.

With further embodiments of the apparatus, the instructions when executed by the processor further cause the apparatus to co-register the angiographic image and the plurality of images.

With further embodiments of the apparatus, the instructions when executed by the processor further cause the apparatus to: identify a start point of a pull-back operation associated with the plurality of images on the vessel represented in the angiographic image; identify an end point of the pull-back operation associated with the plurality of images on the vessel represented in the angiographic image; and identify a centerline of the vessel between start point and the end point.

With further embodiments of the apparatus, the instructions when executed by the processor further cause the apparatus to identify a plurality of side branches of the vessel on the angiographic image and in the plurality of images; and match a one of the plurality of side branches identified on the angiographic image with a one of the plurality of side branches identified in the plurality of images.

With further embodiments of the apparatus, the instructions when executed by the processor further cause the apparatus to map frames of the plurality of images with locations along the centerline of the vessel on the angiographic image.

With further embodiments of the apparatus, the instructions when executed by the processor further cause the apparatus to generate assessments of the vessel, wherein the assessments comprise a diameter of the vessel, an area of the vessel, or a diameter and area of the vessel and wherein the assessments comprise a diameter of the lumen, an area of the lumen, or a diameter and area of the lumen.

With further embodiments of the apparatus, the instructions when executed by the processor further cause the apparatus to receive an indication of an additional physiological characteristic of the vessel of the patient; and generate the 3D model of the physiology of the vessel from the angiographic image, the plurality of images, and the additional physiological characteristic of the vessel, wherein the additional physiological characteristic of the vessel comprises pressure or flow.

With further embodiments of the apparatus, the instructions when executed by the processor further cause the apparatus to generate an inference of the 3D model of the physiologic of the vessel from a machine learning (ML) model based in part on applying the angiographic image and the plurality of images as inputs to the ML model.

With further embodiments of the apparatus, the ML model is trained based in part on a supervised learning training algorithm with expected outputs of the ML model derived based on a computation fluid dynamics (CFD) model, wherein the CFD model takes an angiographic image and a plurality of images as input and generates a 3D vessel physiology model as output.

In some embodiments, the disclosure can be implemented as a computer-readable storage device. The storage device can include instructions executable by a processor of a computing device coupled to an intravascular imaging device and a fluoroscope device, wherein when executed, the instructions cause the computing device to: receive, from the fluoroscope device, an angiographic image if a vessel of a patient; receive, from the intravascular imaging device, a plurality of images associated with the vessel of the patient, the plurality of images comprising multidimensional and multivariate images; and generate a three-dimensional (3D) model of a physiology of the vessel from the angiographic image and the plurality of images.

With further embodiments of the storage device, the instructions when executed by the processor further cause the computing device to generate a graphical information element comprising an indication of the 3D model; and cause the graphical information element to be displayed on a display coupled to the computing device.

With further embodiments of the storage device, the instructions when executed by the processor further cause the computing device to identify a start point of a pull-back operation associated with the plurality of images on the vessel represented in the angiographic image; identify an end point of the pull-back operation associated with the plurality of images on the vessel represented in the angiographic image; identify a centerline of the vessel between start point and the end point; identify a plurality of side branches of the vessel on the angiographic image and in the plurality of images; match a one of the plurality of side branches identified on the angiographic image with a one of the plurality of side branches identified in the plurality of images; and map frames of the plurality of images with locations along the centerline of the vessel on the angiographic image.

With further embodiments of the storage device, the instructions when executed by the processor further cause the computing device to generate assessments of the vessel, wherein the assessments comprise a diameter of the vessel, an area of the vessel, or a diameter and area of the vessel and wherein the assessments comprise a diameter of the lumen, an area of the lumen, or a diameter and area of the lumen.

With further embodiments of the storage device, the instructions when executed by the processor further cause the computing device to receive an indication of an additional physiological characteristic of the vessel of the patient; and generate the 3D model of the physiology of the vessel from the angiographic image, the plurality of images, and the additional physiological characteristic of the vessel, wherein the additional physiological characteristic of the vessel comprises pressure or flow.

With further embodiments of the storage device, the instructions when executed by the processor further cause the computing device to generate an inference of the 3D model of the physiologic of the vessel from a machine learning (ML) model based in part on applying the angiographic image and the plurality of images as inputs to the ML model, wherein the ML model is trained based in part on a supervised learning training algorithm with expected outputs of the ML model derived based on a computation fluid dynamics (CFD) model, wherein the CFD model takes an angiographic image and a plurality of images as input and generates a 3D vessel physiology model as output.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates an intravascular treatment system in accordance with at least one embodiment.

FIG. 2A illustrates another intravascular treatment system in accordance with at least another embodiment.

FIG. 2B illustrates a portion of the intravascular treatment system of FIG. 2A.

FIG. 2C illustrates a portion of the intravascular treatment system of FIG. 2A.

FIG. 3 illustrates a routine 300 for generating a three-dimensional (3D) model of a vessel in accordance with at least one embodiment.

FIG. 4A, 4B, 4C, 4D, 4E, 4F, 4G, and 4H illustrates images of elements or features of the subject matter in accordance with at least one embodiment.

FIG. 5 illustrates an example machine learning (ML) environment in accordance with at least one embodiment.

FIG. 6 illustrates a computer-readable storage medium in accordance with at least one embodiment.

FIG. 7 illustrates a diagrammatic representation of a machine in the form of a computer system within which a set of instructions may be executed for causing the machine to perform any one or more of the methodologies discussed herein.

### DETAILED DESCRIPTION

As introduced above, in an exemplary embodiment, a system is arranged to generate a 3D model of a vessel physiology from a series of intravascular ultrasound (IVUS) images co-registered with a single angiographic image. In some embodiments, the system can be arranged to further utilize an additional vessel characteristic (e.g., pressure measurement, flow measurements, or the like to generate the model. As a specific example, the present disclosure provides to generate a 3D model of a vessel physiology from the series of IVUS images co-registered with a single angiographic image and a measurement of aortic pressure (e.g., Pa, FFR, or the like). Although the disclosure uses examples of the aortic and coronary arteries, the disclosed system and methods can be implemented to generate a 3D model of other types of vessels.

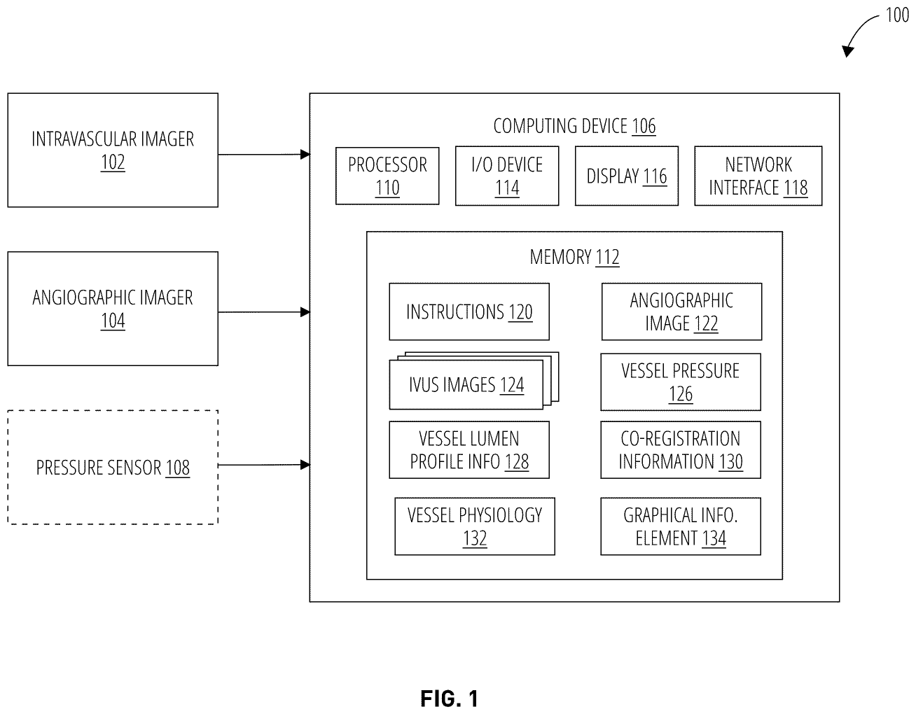

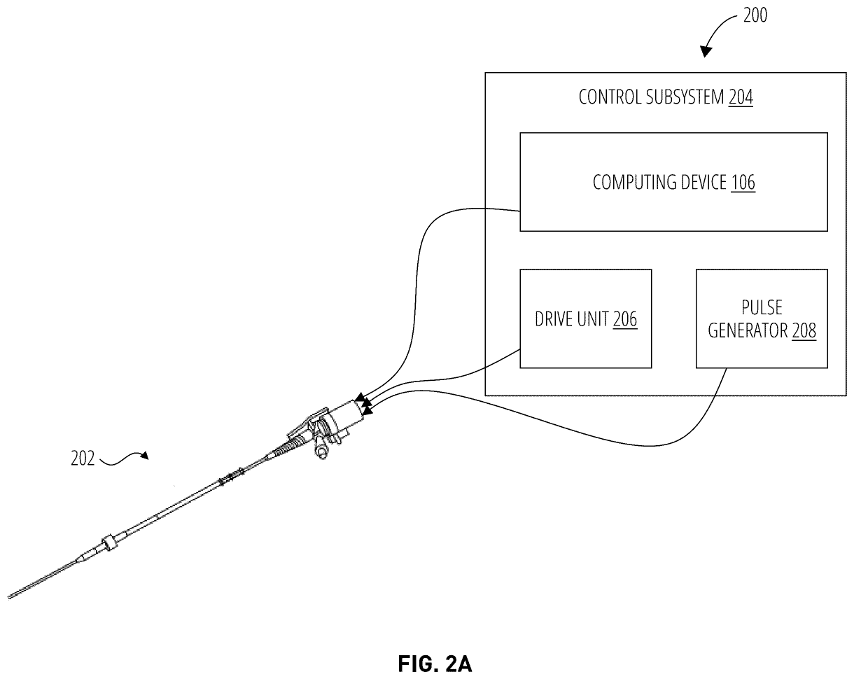

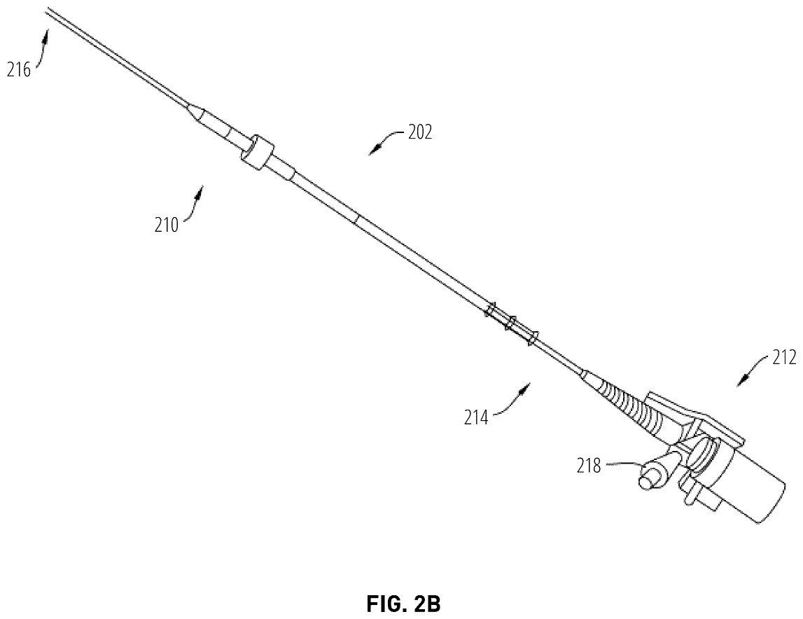

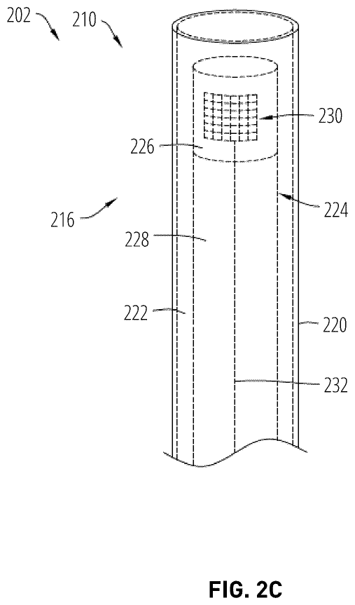

FIG. 1 illustrates a vessel physiology modeling system 100, in accordance with an embodiment of the present disclosure. In general, vessel physiology modeling system 100 is a system for generating a virtual model of a vessel based on various images and characteristics of the vessel. To that end, vessel physiology modeling system 100 includes intravascular imager 102, angiographic imager 104, computing device 106, and optionally pressure sensor 108. Intravascular imager 102 can any of a variety of intravascular imagers (e.g., IVUS, OCT, OCE, or the like). In a specific example, the intravascular imager 102 can be the intravascular treatment system 200 described with reference to FIG. 2A below. Likewise, the angiographic imager 104 can be any of a variety of angiographic images (e.g., a fluoroscope machine, or the like). Additionally, pressure sensor 108 can be any of a variety of vessel pressure sensing devices (e.g., pressure sensing catheter, or the like). With some embodiments, intravascular imager 102 and pressure sensor 108 can be integrated into the same device.

Computing device 106 can be any of a variety of computing devices. In some embodiments, computing device 106 can be incorporated into and/or implemented by a console of intravascular imager 102. With some embodiments, computing device 106 can be a workstation or server communicatively coupled to intravascular imager 102. With still other embodiments, computing device 106 can be provided by a cloud based computing device, such as, by a computing as a service system accessibly over a network (e.g., the Internet, an intranet, a wide area network, or the like). Computing device 106 can include processor 110, memory 112, input and/or output (I/O) device 114, and network interface 118.

The processor 110 may include circuity or processor logic, such as, for example, any of a variety of commercial processors. In some examples, processor 110 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 110 may include graphics processing portions and may include dedicated memory, multiple-threaded processing and/or some other parallel processing capability. In some examples, the processor 110 may be an application specific integrated circuit (ASIC) or a field programmable integrated circuit (FPGA).

The memory 112 may include logic, a portion of which includes arrays of integrated circuits, forming non-volatile memory to persistently store data or a combination of non-volatile memory and volatile memory. It is to be appreciated, that the memory 112 may be based on any of a variety of technologies. In particular, the arrays of integrated circuits included in memory 112 may be arranged to form one or more types of memory, such as, for example, dynamic random access memory (DRAM), NAND memory, NOR memory, or the like.

I/O devices 114 can be any of a variety of devices to receive input and/or provide output. For example, I/O devices 114 can include, a keyboard, a mouse, a joystick, a foot pedal, a haptic feedback device, an LED, or the like. Display 116 can be a conventional display or a touch-enabled display. Further, display 116 can utilize a variety of display technologies, such as, liquid crystal display (LCD), light emitting diode (LED), or organic light emitting diode (OLED), or the like.

Network interface 118 can include logic and/or features to support a communication interface. For example, network interface 118 may include one or more interfaces that operate according to various communication protocols or standards to communicate over direct or network communication links. Direct communications may occur via use of communication protocols or standards described in one or more industry standards (including progenies and variants). For example, network interface 118 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 118 can include logic and/or features to enable communication over a variety of wired or wireless network standards (e.g., 802.11 communication standards). For example, network interface 118 may be arranged to support wired communication protocols or standards, such as, Ethernet, or the like. As another example, network interface 118 may be arranged to support wireless communication protocols or standards, such as, for example, Wi-Fi, Bluetooth, ZigBee, LTE, 5G, or the like.

Memory 112 can include instructions 120, angiographic image 122, IVUS images 124, vessel pressure 126, vessel lumen profile information 128, co-registration information 130, vessel physiology 132, and graphical information element 134.

During operation, processor 110 can execute instructions 120 to cause computing device 106 to receive IVUS images 124 from intravascular imager 102. In general, IVUS images 124 are multi-dimensional multivariate images comprising indications of the vessel type, a lesion in the vessel, the lesion type, stent detection, the lumen border, the lumen dimensions, the minimum lumen area (MLA), the media border (e.g., a media border for media within the blood vessel), the media dimensions, the calcification angle/arc, the calcification coverage, combinations thereof, and/or the like.

Processor 110 can further execute instructions 120 to cause computing device 106 to receive angiographic image 122 from angiographic imager 104. In general, angiographic image 122 is an x-ray image of the blood vessels in a patient's heart. A contrast agent is injected (e.g., via a catheter, or the like) into a vessel and an x-ray image is captured while the contrast is active thereby making the vessel visible to the x-ray.

Optionally, processor 110 can further execute instructions 120 to cause computing device 106 to receive vessel pressure 126. With some embodiments, processor 110 can execute instructions 120 to cause computing device 106 to receive vessel pressure 126 automatically (e.g., from pressure sensor 108, or the like). In other embodiments, processor 110 can execute instructions 120 to cause computing device 106 from a user of vessel physiology modeling system 100. For example, a physician can input the vessel pressure 126 using I/O device 114.

Processor 110 can further execute instructions 120 to cause computing device 106 to determine vessel lumen profile information 128 from IVUS images 124. For example, processor 110 can execute instructions 120 to automatically determine the lumen area at various points along the vessel from IVUS images 124. As another example, processor 110 can execute instructions 120 to automatically determine the vessel border at various points along the vessel from IVUS images 124. As another example, processor 110 can execute instructions 120 to automatically determine the plaque burden of the vessel at various points along the vessel from IVUS images 124. These are just a few examples of assessments that can be represented in vessel lumen profile information 128.

Processor 110 can further execute 120 to cause computing device 106 to co-register the angiographic image 122 and IVUS images 124. It is to be appreciated that IVUS images 124 is a series of multiple cross sectional views of a vessel acquired by during a pull-back of an intra-coronary ultrasound transducer (e.g., intravascular imager 102) delineating its lumen and arterial wall; while angiographic image 122 is an image captured by an x-ray beam (e.g., angiographic imager 104) emitted at the vessel during a period when contract media is injected into the vessel thereby outlining an intra-luminal silhouette of the vessel. As such angiographic image 122 and IVUS images 124 are complimentary. However, given they are captured by different pieces of equipment (e.g., intravascular imager 102 and angiographic imager 104, or the like) the locations of the captured IVUS images 124 are not correlated with locations on the angiographic image 122. Accordingly, a process to register or map the IVUS images 124 to locations on angiographic image 122 is provided and referred to herein as co-registration.

Several co-registration processes are available. As such, a complete discussion of co-registration procedures is not provided herein. However, in general processor 110 can execute instructions 120 to receive (or determine) a location of the start and end of the “pull-back” operation resulting in the IVUS images 124 on the angiographic image 122. Further, processor 110 can execute instructions 120 to determine the location of landmarks (e.g., side-branches, or the like) in both the IVUS images 124 and angiographic image 122 and to map the locations to each other, resulting in co-registration information 130.

Processor 110 can further execute instructions 120 to cause computing device 106 to generate vessel physiology 132 from angiographic image 122 and IVUS images 124. With some embodiments, processor 110 can execute instructions 120 to cause computing device 106 to generate vessel physiology 132 from angiographic image 122, IVUS images 124, and vessel pressure 126. Processor 110 can execute instructions 120 to generate vessel physiology 132 from angiographic image 122, IVUS images 124, vessel lumen profile information 128, co-registration information 130, and optionally, vessel pressure 126. With some embodiments, vessel physiology 132 is a 3D model of the physiology of the vessel represented in the IVUS images 124 and captured on angiographic image 122.

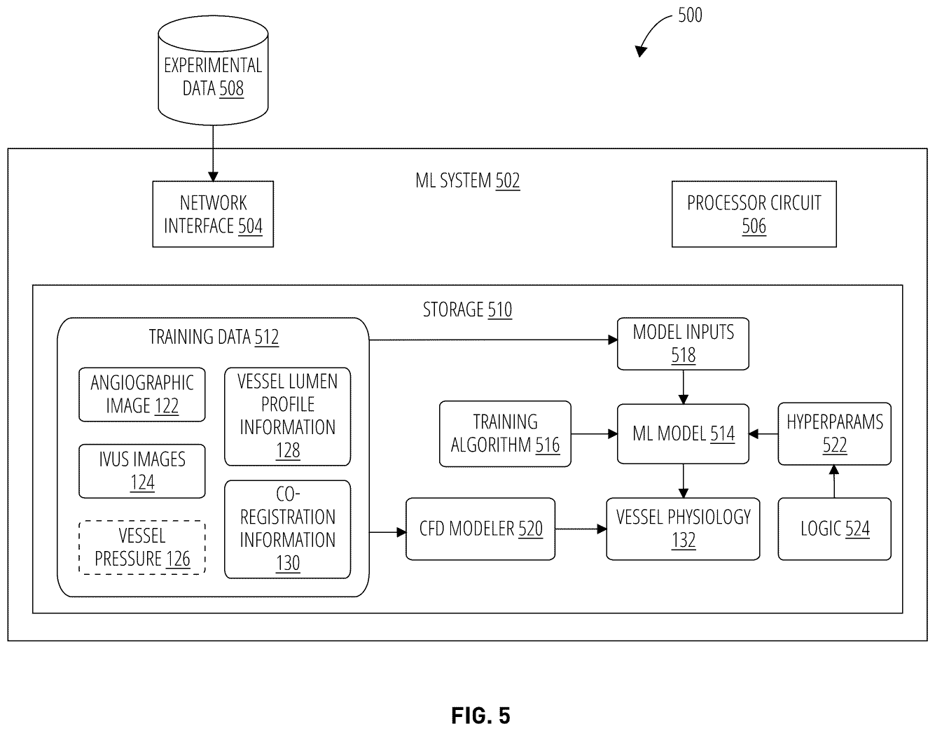

With some embodiments, processor 110 can execute instructions 120 to generate vessel physiology 132 from angiographic image 122, IVUS images 124, vessel lumen profile information 128, co-registration information 130, and optionally, vessel pressure 126 using a machine learning model (e.g., a neural network (NN), a convolutional neural network (CNN), a random forest model, or the like). In other examples, processor 110 can execute instructions 120 to generate vessel physiology 132 using a numerical analysis model, such as a computational fluid dynamics (CFD) model. The processor 110 can execute instructions 120 to generate vessel physiology 132 from a machine learning model trained using inputs described herein and an expected output of a vessel physiology model generated using a CFD model. This is described in greater detail below, for example, with respect to FIG. 5.

Additionally, in some embodiments, processor 110 can execute instructions 120 to generate a graphical information element 134 comprising indications of vessel physiology 132 and cause the graphical information element 134 to be displayed for a user on display 116.

FIG. 2A, FIG. 2B, and FIG. 2C illustrate an example intravascular treatment system 200 and are described together herein. FIG. 2A is a component level view while FIG. 2B and FIG. 2C are side and perspective views, respectively, of a portion of the intravascular treatment system 200 of FIG. 2A. The intravascular treatment system 200 takes the form of an IVUS imaging system and can be implemented as part of the vessel physiology modeling system 100 of FIG. 1. The intravascular treatment system 200 includes a catheter 202 and a control subsystem 204. The control subsystem 204 includes the computing device 106, a drive unit 206 and a pulse generator 208. The catheter 202 and control subsystem 204 are operably coupled, or more specifically, the catheter 202 is electrically and/or mechanically coupled to the computing device 106, drive unit 206, and pulse generator 208 such that signals (e.g., control, measurement, image data, or the like) can be communicated between the catheter 202 and control subsystem 204.

It is noted that the computing device 106 includes display 116. However, in some applications, display 116 may be provided as a separate unit from computing device 106, for example, in a different housing, or the like. In some instances, the pulse generator 208 forms electric pulses that may be input to one or more transducers 230 disposed in the catheter 202.

In some instances, mechanical energy from the drive unit 206 may be used to drive an imaging core 224 disposed in the catheter 202. In some instances, electric signals transmitted from the one or more transducers 230 may be input to the processor 110 of computing device 106 for processing as outlined here. For example, to be used to generate vessel lumen profile information 128 and graphical information element 134. In some instances, the processed electric signals from the one or more transducers 230 can also be displayed as one or more images on the display 116.

In some instances, the processor 110 may also be used to control the functioning of one or more of the other components of control subsystem 204. For example, the processor 110 may be used to control at least one of the frequency or duration of the electrical pulses transmitted from the pulse generator 208, the rotation rate of the imaging core 224 by the drive unit 206, the velocity or length of the pullback of the imaging core 224 by the drive unit 206, or one or more properties of one or more images formed on the display 116, such as, the vessel lumen profile information 128 and graphical information element 134.

FIG. 2B is a side view of one embodiment of the catheter 202 of the intravascular treatment system 200 of FIG. 2A. The catheter 202 includes an elongated member 210 and a hub 212. The elongated member 210 includes a proximal end 214 and a distal end 216. In FIG. 2B, the proximal end 214 of the elongated member 210 is coupled to the catheter hub 212 and the distal end 216 of the elongated member 210 is configured and arranged for percutaneous insertion into a patient. Optionally, the catheter 202 may define at least one flush port, such as flush port 218. The flush port 218 may be defined in the hub 212. The hub 212 may be configured and arranged to couple to the control subsystem 204 of intravascular treatment system 200. In some instances, the elongated member 210 and the hub 212 are formed as a unitary body. In other instances, the elongated member 210 and the catheter hub 212 are formed separately and subsequently assembled.

FIG. 2C is a perspective view of one embodiment of the distal end 216 of the elongated member 210 of the catheter 202. The elongated member 210 includes a sheath 220 with a longitudinal axis (e.g., a central longitudinal axis extending axially through the center of the sheath 220 and/or the catheter 202) and a lumen 222. An imaging core 224 is disposed in the lumen 222. The imaging core 224 includes an imaging device 226 coupled to a distal end of a driveshaft 228 that is rotatable either manually or using a computer-controlled drive mechanism. One or more transducers 230 may be mounted to the imaging device 226 and employed to transmit and receive acoustic signals. The sheath 220 may be formed from any flexible, biocompatible material suitable for insertion into a patient. Examples of suitable materials include, for example, polyethylene, polyurethane, plastic, spiral-cut stainless steel, nitinol hypotube, and the like or combinations thereof.

In some instances, for example as shown in these figures, an array of transducers 230 are mounted to the imaging device 226. Alternatively, a single transducer may be employed. Any suitable number of transducers 230 can be used. For example, there can be two, three, four, five, six, seven, eight, nine, ten, twelve, fifteen, sixteen, twenty, twenty-five, fifty, one hundred, five hundred, one thousand, or more transducers. As will be recognized, other numbers of transducers may also be used. When a plurality of transducers 230 are employed, the transducers 230 can be configured into any suitable arrangement including, for example, an annular arrangement, a rectangular arrangement, or the like.

The one or more transducers 230 may be formed from materials capable of transforming applied electrical pulses to pressure distortions on the surface of the one or more transducers 230, and vice versa. Examples of suitable materials include piezoelectric ceramic materials, piezocomposite materials, piezoelectric plastics, barium titanates, lead zirconate titanates, lead metaniobates, polyvinylidene fluorides, and the like. Other transducer technologies include composite materials, single-crystal composites, and semiconductor devices (e.g., capacitive micromachined ultrasound transducers (“cMUT”), piezoelectric micromachined ultrasound transducers (“pMUT”), or the like).

The pressure distortions on the surface of the one or more transducers 230 form acoustic pulses of a frequency based on the resonant frequencies of the one or more transducers 230. The resonant frequencies of the one or more transducers 230 may be affected by the size, shape, and material used to form the one or more transducers 230. The one or more transducers 230 may be formed in any shape suitable for positioning within the catheter 202 and for propagating acoustic pulses of a desired frequency in one or more selected directions. For example, transducers may be disc-shaped, block-shaped, rectangular-shaped, oval-shaped, and the like. The one or more transducers may be formed in the desired shape by any process including, for example, dicing, dice and fill, machining, microfabrication, and the like.

As an example, each of the one or more transducers 230 may include a layer of piezoelectric material sandwiched between a matching layer and a conductive backing material formed from an acoustically absorbent material (e.g., an epoxy substrate with tungsten particles). During operation, the piezoelectric layer may be electrically excited to cause the emission of acoustic pulses.

The one or more transducers 230 can be used to form a radial cross-sectional image of a surrounding space. Thus, for example, when the one or more transducers 230 are disposed in the catheter 202 and inserted into a blood vessel of a patient, the one more transducers 230 may be used to form an image of the walls of the blood vessel and tissue surrounding the blood vessel.

The imaging core 224 is rotated about the longitudinal axis of the catheter 202. As the imaging core 224 rotates, the one or more transducers 230 emit acoustic signals in different radial directions (e.g., along different radial scan lines). For example, the one or more transducers 230 can emit acoustic signals at regular (or irregular) increments, such as 256 radial scan lines per revolution, or the like. It will be understood that other numbers of radial scan lines can be emitted per revolution, instead.

When an emitted acoustic pulse with sufficient energy encounters one or more medium boundaries, such as one or more tissue boundaries, a portion of the emitted acoustic pulse is reflected to the emitting transducer as an echo pulse. Each echo pulse that reaches a transducer with sufficient energy to be detected is transformed to an electrical signal in the receiving transducer. The one or more transformed electrical signals are transmitted to the processor 110 of the computing device 106 where it is processed to form IVUS images 124 and subsequently generate vessel lumen profile information 128 and graphical information element 134 to be displayed on display 116. In some instances, the rotation of the imaging core 224 is driven by the drive unit 206, which can be disposed in control subsystem 204. In alternate embodiments, the one or more transducers 230 are fixed in place and do not rotate. In which case, the driveshaft 228 may, instead, rotate a mirror that reflects acoustic signals to and from the fixed one or more transducers 230.

When the one or more transducers 230 are rotated about the longitudinal axis of the catheter 202 emitting acoustic pulses, a plurality of images can be formed that collectively form a radial cross-sectional image (e.g., a tomographic image) of a portion of the region surrounding the one or more transducers 230, such as the walls of a blood vessel of interest and tissue surrounding the blood vessel. The radial cross-sectional image can form the basis of IVUS images 124 and can optionally be displayed on display 116. The at least one of the imaging core 224 can be either manually rotated or rotated using a computer-controlled mechanism.

The imaging core 224 may also move longitudinally along the blood vessel within which the catheter 202 is inserted so that a plurality of cross-sectional images may be formed along a longitudinal length of the blood vessel. During an imaging procedure the one or more transducers 230 may be retracted (e.g., pulled back) along the longitudinal length of the catheter 202. The catheter 202 can include at least one telescoping section that can be retracted during pullback of the one or more transducers 230. In some instances, the drive unit 206 drives the pullback of the imaging core 224 within the catheter 202. The drive unit 206 pullback distance of the imaging core can be any suitable distance including, for example, at least 5 cm, 10 cm, 15 cm, 20 cm, 25 cm, or more. The entire catheter 202 can be retracted during an imaging procedure either with or without the imaging core 224 moving longitudinally independently of the catheter 202.

A stepper motor may, optionally, be used to pull back the imaging core 224. The stepper motor can pull back the imaging core 224 a short distance and stop long enough for the one or more transducers 230 to capture an image or series of images before pulling back the imaging core 224 another short distance and again capturing another image or series of images, and so on.

The quality of an image produced at different depths from the one or more transducers 230 may be affected by one or more factors including, for example, bandwidth, transducer focus, beam pattern, as well as the frequency of the acoustic pulse. The frequency of the acoustic pulse output from the one or more transducers 230 may also affect the penetration depth of the acoustic pulse output from the one or more transducers 230. In general, as the frequency of an acoustic pulse is lowered, the depth of the penetration of the acoustic pulse within patient tissue increases. In some instances, the intravascular treatment system 200 operates within a frequency range of 5 MHz to 200 MHZ.

One or more conductors 232 can electrically couple the transducers 230 to the control subsystem 204. In which case, the one or more conductors 232 may extend along a longitudinal length of the rotatable driveshaft 228.

The catheter 202 with one or more transducers 230 mounted to the distal end 216 of the imaging core 224 may be inserted percutaneously into a patient via an accessible blood vessel, such as the femoral artery, femoral vein, or jugular vein, at a site remote from the selected portion of the selected region, such as a blood vessel, to be imaged. The catheter 202 may then be advanced through the blood vessels of the patient to the selected imaging site, such as a portion of a selected blood vessel.

An image or image frame (“frame”) can be generated each time one or more acoustic signals are output to surrounding tissue and one or more corresponding echo signals are received by the imaging device 226 and transmitted to the processor 110 of the computing device 106. Alternatively, an image or image frame can be a composite of scan lines from a full or partial rotation of the imaging core or device. A plurality (e.g., a sequence) of frames may be acquired over time during any type of movement of the imaging device 226. For example, the frames can be acquired during rotation and pullback of the imaging device 226 along the target imaging location. It will be understood that frames may be acquired both with or without rotation and with or without pullback of the imaging device 226. Moreover, it will be understood that frames may be acquired using other types of movement procedures in addition to, or in lieu of, at least one of rotation or pullback of the imaging device 226.

In some instances, when pullback is performed, the pullback may be at a constant rate, thus providing a tool for potential applications able to compute longitudinal vessel/plaque measurements. In some instances, the imaging device 226 is pulled back at a constant rate of about 0.3-0.9 mm/s or about 0.5-0.8 mm/s. In some instances, the imaging device 226 is pulled back at a constant rate of at least 0.3 mm/s. In some instances, the imaging device 226 is pulled back at a constant rate of at least 0.4 mm/s. In some instances, the imaging device 226 is pulled back at a constant rate of at least 0.5 mm/s. In some instances, the imaging device 226 is pulled back at a constant rate of at least 0.6 mm/s. In some instances, the imaging device 226 is pulled back at a constant rate of at least 0.7 mm/s. In some instances, the imaging device 226 is pulled back at a constant rate of at least 0.8 mm/s.

In some instances, the one or more acoustic signals are output to surrounding tissue at constant intervals of time. In some instances, the one or more corresponding echo signals are received by the imaging device 226 and transmitted to the processor 110 of the computing device 106 at constant intervals of time. In some instances, the resulting frames are generated at constant intervals of time.

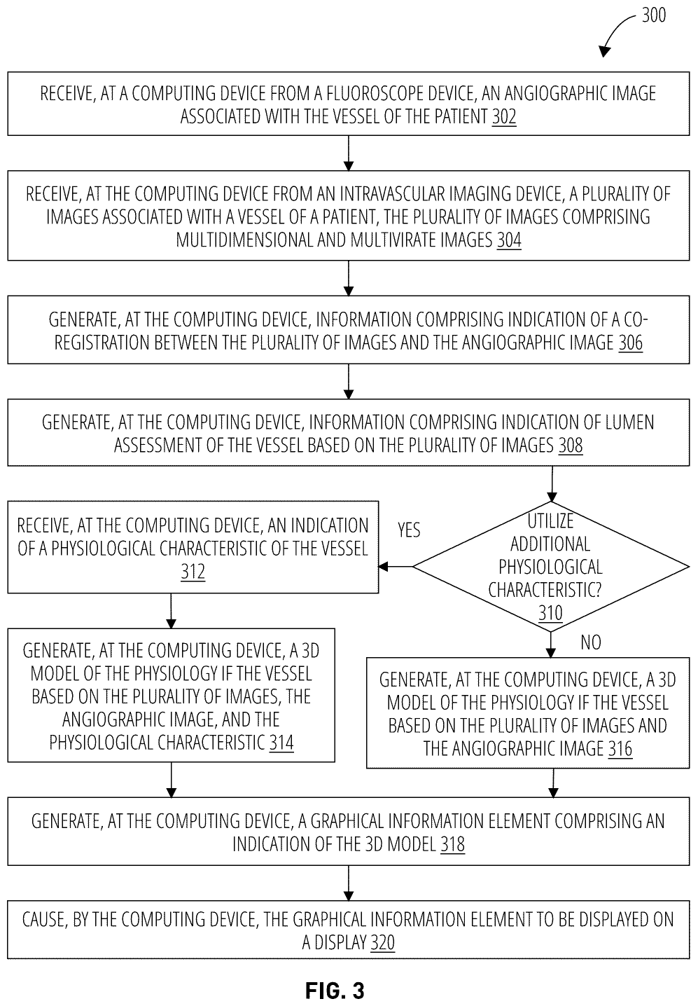

FIG. 3 illustrates routine 300 according to some embodiments of the present disclosure. Routine 300 can be implemented by vessel physiology modeling system 100 or another computing device as outlined herein to provide a 3D physiological representation or a vessel from a single angiographic image, a series of intravascular images, and a pressure or flow.

Routine 300 can begin at block 302. At block 302 “receive, at a computing device from a fluoroscope device, an angiographic image associated with the vessel of the patient” computing device 106 of vessel physiology modeling system 100 receives angiographic image 122 from angiographic imager 104 where angiographic image 122 is an angiographic image captured by an x-ray machine while the vessel is exposed to a contrast media. For example, processor 110 can execute instructions 120 to receive data including indications of angiographic image 122 from angiographic imager 104 via network interface 118.

Continuing to block 304 “receive, at the computing device from an intravascular imaging device, a plurality of images associated with a vessel of a patient, the plurality of images comprising multidimensional and multivariate images” computing device 106 of vessel physiology modeling system 100 receives IVUS images 124 from intravascular imager 102 where IVUS images 124 is multidimensional and multivariate images of the vessel. For example, processor 110 can execute instructions 120 to receive data including indications of IVUS images 124 from intravascular imager 102 via network interface 118.

Continuing to block 306 “generate, at the computing device, information comprising indication of a co-registration between the plurality of images and the angiographic image” information comprising indications of a mapping or registration between a portion of a vessel represented in the image received at block 302 and the images of the portion of the vessel received at block 304 can be generated. For example, processor 110 can execute instructions 120 to identify a start and end point of the pull-back operation through the vessel on the angiographic image 122. Further, processor 110 can execute instructions 120 to identify one or more other landmarks (e.g., vessel centerline, side branches, or the like) in both the angiographic image 122 and IVUS images 124 and can map or coordinate the frames of IVUS images 124 with locations on angiographic image 122 based on the identified start and end point as well as the other landmarks. As noted above, co-registration is a complex process and specific details of actual co-registration are beyond the scope of this description. Further, processor 110 can execute instructions 120 to store the indications of the co-registration as co-registration information 130.

Continuing to block 308 “generate, at the computing device, information comprising indication of lumen assessment of the vessel based on the plurality of images” information comprising indications of a lumen assessment of the vessel based on the images received at block 304 can be generated. For example, processor 110 can execute instructions 120 to automatically identify geometric characteristics of the vessel and lumen (e.g., diameter, area, etc.) represented in the images received at blocks 302 and 304 and store the indications as vessel lumen profile information 128. With some embodiments, processor 110 can execute instructions 120 to generate the lumen and vessel assessments based on machine learning, image processing, geometric image analysis, or the like.

Continuing to block 310 “utilize additional physiological characteristic?” computing device 106 of vessel physiology modeling system 100 can determine whether to utilize an additional physiological characteristic in the 3D model generation process (e.g., as outlined above). From block 310, method 300 can continue to either block 312 or block 316. Method 300 can continue from block 310 to block 312 based on a determination at block 310 that an additional physiological characteristic will be utilized in generating the 3D model of the vessel physiology; while the method 300 can continue from block 310 to block 316 based on a determination at block 310 that an additional physiological characteristic will not be utilized in generating the 3D model of the vessel physiology.

At block 312 “receive, at the computing device, an indication of a physiological characteristic of the vessel” computing device 106 of vessel physiology modeling system 100 receives vessel pressure 126. For example, processor 110 can execute instructions 120 to receive vessel pressure 126 automatically from pressure sensor 108. As another example, processor 110 can execute instructions 120 to receive vessel pressure 126 from a user via I/O device 114, or the like. With some embodiments, vessel pressure 126 can comprise an aortic pressure. In other embodiments, vessel pressure 126 can comprise a blood flow velocity measurement. With some embodiments, vessel pressure 126 can comprise an aortic pressure measurement taken during the pull-back operation associated with the IVUS images 124 (e.g., FFR, DFR, or the like).

Continuing from block 312 to block 314 “generate, at the computing device, a 3D model of the physiology if the vessel based on the plurality of images, the angiographic image, and optionally the physiological characteristic” a 3D model of the physiology of the vessel represented in angiographic image 122 and IVUS images 124 can be generated. For example, processor 110 can execute instructions 120 to generate a 3D model, or representation, of the physiology of the vessel (or portion of vessel) represented in IVUS images 124 and depicted in 2D form on angiographic image 122 and store indications of the 3D model as vessel physiology 132. Processor 110 can execute instructions 120 to generate the 3D model using vessel lumen profile information 128 and co-registration information 130 as well as another physiological characteristic (e.g., vessel pressure 126, or the like). More particularly, processor 110 can execute instructions 120 to generate the 3D representation from the generated lumen diameters, the generated centerline and the 2D representation of the vessel depicted in angiographic image 122. Specifically, given the mapping between IVUS images 124 and angiographic image 122 from co-registration information 130, the lumen diameters of vessel lumen profile information 128 can be used to form the 3D model indicated by vessel physiology 132.

At block 316 “generate, at the computing device, a 3D model of the physiology if the vessel based on the plurality of images and the angiographic image” a 3D model of the physiology of the vessel represented in angiographic image 122 and IVUS images 124 can be generated. For example, processor 110 can execute instructions 120 to generate a 3D model, or representation, of the physiology of the vessel (or portion of vessel) represented in IVUS images 124 and depicted in 2D form on angiographic image 122 and store indications of the 3D model as vessel physiology 132. Processor 110 can execute instructions 120 to generate the 3D model using vessel lumen profile information 128 and co-registration information 130. More particularly, processor 110 can execute instructions 120 to generate the 3D representation from the generated lumen diameters, the generated centerline and the 2D representation of the vessel depicted in angiographic image 122. Specifically, given the mapping between IVUS images 124 and angiographic image 122 from co-registration information 130, the lumen diameters of vessel lumen profile information 128 can be used to form the 3D model indicated by vessel physiology 132.

Method 300 can continue from blocks 314 and 316 6 to block 318. At block 318 “generate, at the computing device, a graphical information element comprising an indication of the 3D model” a graphical information element comprising an indication of vessel physiology 132 (e.g., the 3D model) can be generated. For example, processor 110 can execute instructions 120 to generate graphical information element 134 comprising indications of the 3D model of the vessel represented in angiographic image 122 and IVUS images 124 and indicated in vessel physiology 132.

Continuing to block 320 “cause, by the computing device, the graphical information element to be displayed on a display” the graphical information element generated at block 314 can be displayed on a display. For example, processor 110 can execute instructions 120 to cause graphical information element 134 to be displayed by display 116.

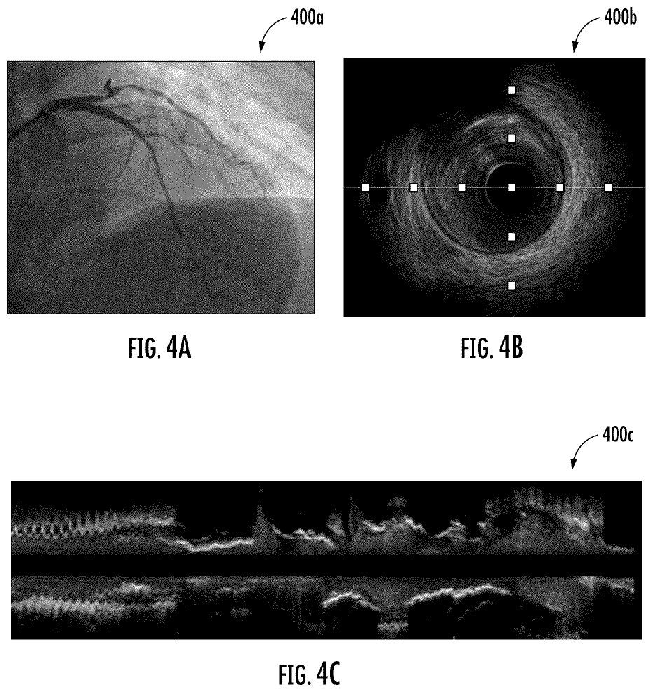

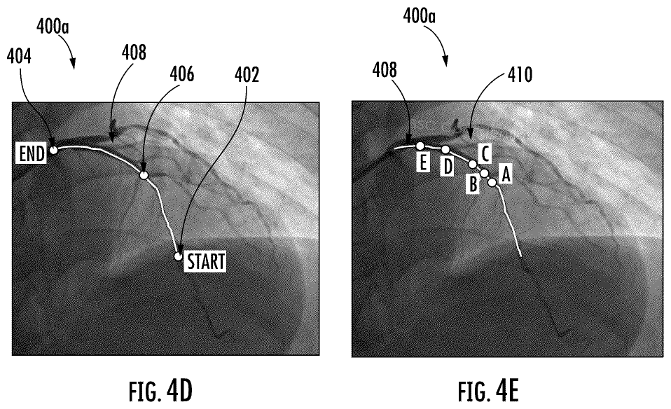

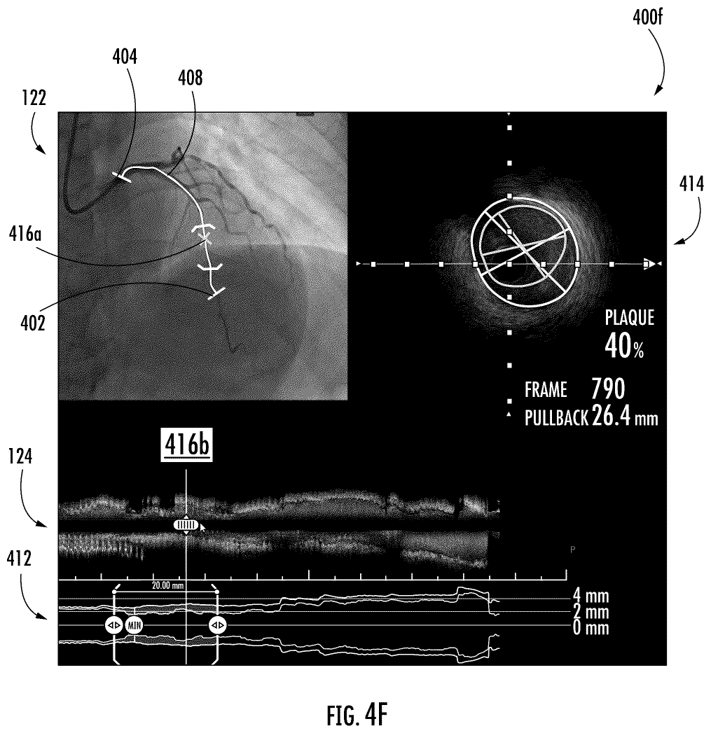

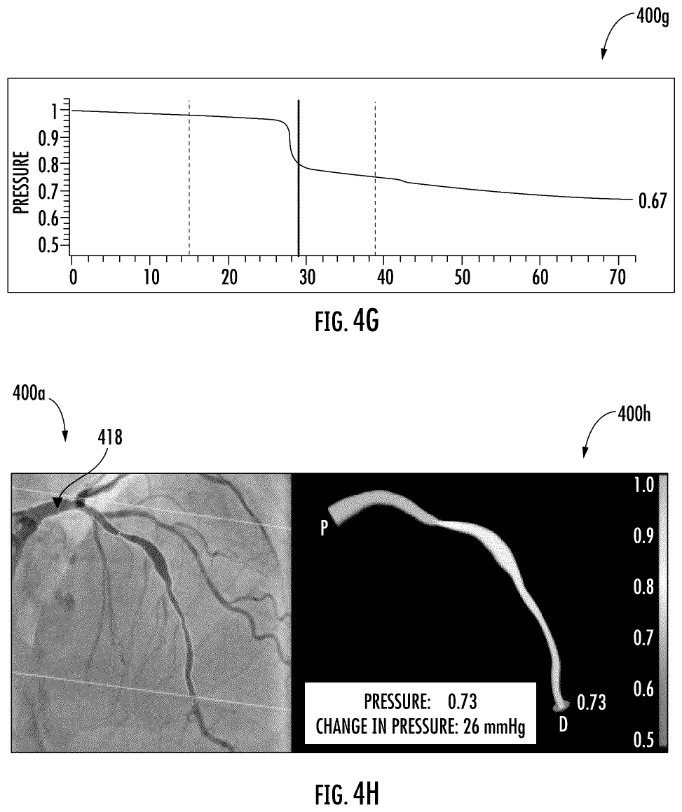

FIG. 4A to FIG. 4H depict examples of images and assessments described herein. These figures are described with reference to the operations or block of routine 300 of FIG. 3. However, it is to be appreciated that this is done for purposes of clarity of presentation and not to be limiting. Turning to FIG. 4A, an angiographic image 400a is depicted. As discussed above vessel physiology modeling system 100 can execute instructions 120 to receive angiographic image 400a (or information elements and/or data structures comprising indications of angiographic image 400a) at block 302.

FIG. 4B and FIG. 4C illustrate on-axis IVUS images view 400b and IVUS images 400c, respectively. For example, on-axis IVUS images view 400b depicts an on-axis (or short axis) view of a vessel at represented by IVUS images 124 at one frame of the IVUS images 124 while IVUS images 400c depicts a longitudinal view of the vessel between the start and end of the pull-back operation which generated IVUS images 124. As discussed above vessel physiology modeling system 100 can execute instructions 120 to receive on-axis IVUS images view 400b and IVUS images 400c (or information elements and/or data structures comprising indications of on-axis IVUS images view 400b and IVUS images 400c) at block 304.

FIG. 4D illustrates angiographic image 400a with start point 402, end point 404, midpoint 406, and centerline 408 of the vessel (or vessel portion) represented in IVUS images 124 designated on angiographic image 400a. As discussed above, routine 300 can include block 306 to generate co-registration information 130 from angiographic image 122 and IVUS images 124. Identification of the start point 402, end point 404, and centerline 408 as well as side branches (refer to FIG. 4E) can be part of the co-registration process implemented at block 306.

FIG. 4E illustrates angiographic image 400a with side branches 410 designated on angiographic image 400a. Five (5) side branches (A, B, C, D, and E) are designated on the centerline 408 of the vessel represented in angiographic image 122 and IVUS images 124. With some examples, processor 110 can execute instructions 120 to receive indications (e.g., via I/O device 114, or the like) of the start point 402, end point 404, and mid-point 406 and generate centerline 408 from the indicated start point 402, end point 404, midpoint 406 and angiographic image 122. As a further example, processor 110 can execute instructions 120 to receive adjustments (e.g., via I/O device 114, or the like) to the generated centerline 408. Further, processor 110 can execute instructions 120 to generate locations of side branches 410. As a further example, processor 110 can execute instructions 120 to receive adjustments (e.g., via I/O device 114, or the like) to the generated locations of the side branches 410.

FIG. 4F illustrates a graphical information element (GUI) 400f showing a representation of the co-registration information 130 and vessel lumen profile information 128 and angiographic image 122 and IVUS images 124. For example, angiographic image 122 and IVUS images 124 are depicted along with an indication of a longitudinal vessel profile view 412 and vessel assessments 414. It is to be appreciated that the frames of IVUS images 124 are mapped to locations along the vessel centerline 408 between start point 402 and end point 404. As such, location 416a is co-registered or co-located with location 416b. Further, vessel assessments 414 (representative of vessel lumen profile information 128 generated at block 308) are shown for a frame of IVUS images 124 corresponding to the locations 416a and 416b.

FIG. 4G illustrates a pressure curve 400g comprising indications of vessel pressure 126. As discussed above, vessel pressure 126 can be received by vessel physiology modeling system 100 at block 310 of routine 300 and can optionally be utilized in generating the 3D representation of the vessel. In this case, the graph depicted in curve 400g shows pressure along the distance of the IVUS pullback corresponding to the series of IVUS images 124.

FIG. 4H illustrates a 3D model 400h of the vessel 418 represented by angiographic image 122 and IVUS images 124. More specifically, in 3D model 400h shows a 3D representation of the 2D view of vessel 418 shown in angiographic image 400a (e.g., angiographic image 122). As can be seen, the physiological representation of 3D model 400h depicts both geometric and pressure gradients of the vessel 418. As outlined above, routine 300 can include block 312 to generate the 3D model 400h from angiographic image 122, IVUS images 124, vessel pressure 126, vessel lumen profile information 128, and co-registration information 130.

As noted, with some embodiments, processor 110 of computing device 106 can execute instructions 120 to generate vessel physiology 132 using a machine learning (ML) model. In such example, the ML model can be stored in memory 112 of computing device 106. It will be appreciated, that prior to being deployed, the ML model is to be trained. FIG. 5 illustrates an ML environment 500, which can be used to train an ML model that may later be used to generate (or infer) vessel physiology 132 as described herein. The ML environment 500 may include an ML system 502, such as a computing device that applies an ML algorithm to learn relationships. In this example, the ML algorithm can learn relationships between a set of inputs (e.g., angiographic image 122, IVUS images 124, vessel lumen profile information 128, co-registration information 130, and optionally vessel pressure 126) and an output (e.g., vessel physiology 132).

The ML system 502 may make use of experimental data 508 gathered during several prior procedures. Experimental data 508 can include an angiographic image 122 and IVUS images 124 for several patients. The experimental data 508 may be collocated with the ML system 502 (e.g., stored in a storage 510 of the ML system 502), may be remote from the ML system 502 and accessed via a network interface 504, or may be a combination of local and remote data.

Experimental data 508 can be used to form training data 512. As noted above, the ML system 502 may include a storage 510, which may include a hard drive, solid state storage, and/or random access memory. The storage 510 may hold training data 512. In general, training data 512 can include information elements or data structures comprising indications of an angiographic image 122 and IVUS images 124 for several patients. In addition, training data 512 can optionally include 126 for the patients. Further, with some embodiments, training data 512 can include vessel lumen profile information 128 and co-registration information 130 for each of the patients. With some embodiments, experimental data 508 includes just the angiographic image 122 and IVUS images 124 for the patients and ML system 502 is configured (e.g., with processor and instructions executable by the processor) to generate vessel lumen profile information 128 and co-registration information 130 from the angiographic image 122 and IVUS images 124 for each patient represented in experimental data 508.

The training data 512 may be applied to train an ML model 514. Depending on the application, different types of models may be used to form the basis of ML model 514. For instance, in the present example, an artificial neural network (ANN) may be particularly well-suited to learning associations between an angiographic image (e.g., angiographic image 122) and IVUS images (e.g., IVUS images 124) and a 3D model of the vessel physiology (e.g., vessel physiology 132). Convoluted neural networks may also be well-suited to this task. Any suitable training algorithm 516 may be used to train the ML model 514. Nonetheless, the example depicted in FIG. 5 may be particularly well-suited to a supervised training algorithm or reinforcement learning training algorithm. For a supervised training algorithm, the ML system 502 may apply the angiographic image 122 and IVUS images 124 (and optionally, vessel pressure 126, vessel lumen profile information 128, and/or co-registration information 130) as model inputs 518, to which an expected output (e.g., vessel physiology 132) generated from the training data 512 using a CFD modeler 520 may be mapped to learn associations between the model inputs 518 and the vessel physiology 132. In a reinforcement learning scenario, training algorithm 516 may attempt to maximize some or all (or a weighted combination) of the model inputs 518 mappings to vessel physiology 132 to produce ML model 514 having the least error. With some embodiments, training data 512 can be split into “training” and “testing” data wherein some subset of the training data 512 can be used to adjust the ML model 514 (e.g., internal weights of the model, or the like) while another, non-overlapping subset of the training data 512 can be used to measure an accuracy of the ML model 514 to infer (or generalize) a vessel physiology 132 from “unseen” training data 512 (e.g., training data 512 not used to train ML model 514).

The ML model 514 may be applied using a processor circuit 506, which may include suitable hardware processing resources that operate on the logic and structures in the storage 510. The training algorithm 516 and/or the development of the trained ML model 514 may be at least partially dependent on hyperparameters 522. In exemplary embodiments, the model hyperparameters 522 may be automatically selected based on hyperparameter optimization logic 524, which may include any known hyperparameter optimization techniques as appropriate to the ML model 514 selected and the training algorithm 516 to be used. In optional, embodiments, the ML model 514 may be re-trained over time, to accommodate new knowledge and/or updated experimental data 508.

Once the ML model 514 is trained, it may be applied (e.g., by the processor circuit 506, by processor 110, or the like) to new input data (e.g., angiographic image 122 and IVUS images 124 captured during a pre-PCI intervention, or the like. This input to the ML model 514 may be formatted according to a predefined model inputs 518 mirroring the way that the training data 512 was provided to the ML model 514. The ML model 514 may generate a vessel physiology 132 which may be, for example, a generalization or inference of the 3D vessel physiology of the vessel represented in angiographic image 122 and IVUS images 124 provided as input to the ML model 514.

The above description pertains to a particular kind of ML system 502, which applies supervised learning techniques given available training data with input/result pairs. However, the present invention is not limited to use with a specific ML paradigm, and other types of ML techniques may be used. For example, in some embodiments the ML system 502 may apply for example, evolutionary algorithms, or other types of ML algorithms and models to generate vessel physiology 132 from angiographic image 122 and IVUS images 124.

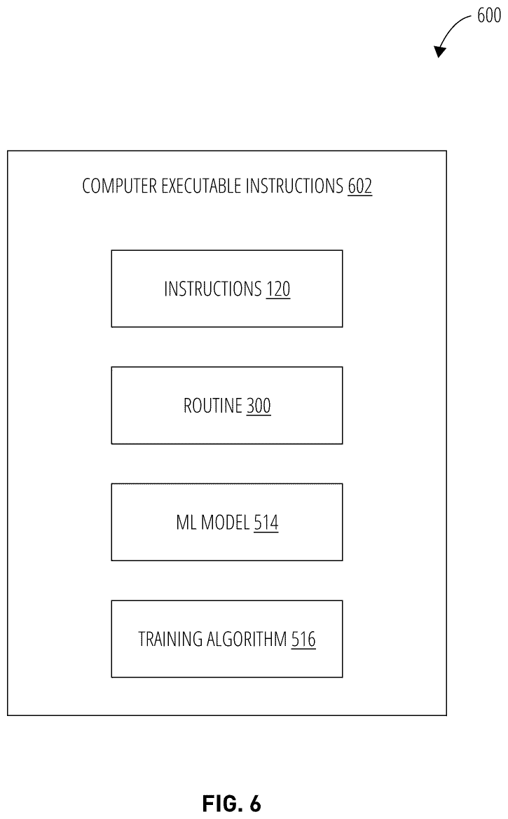

FIG. 6 illustrates computer-readable storage medium 600. Computer-readable storage medium 600 may comprise any non-transitory computer-readable storage medium or machine-readable storage medium, such as an optical, magnetic or semiconductor storage medium. In various embodiments, computer-readable storage medium 600 may comprise an article of manufacture. In some embodiments, computer-readable storage medium 600 may store computer executable instructions 602 with which circuitry (e.g., processor 110, or the like) can execute. For example, computer executable instructions 602 can include instructions to implement operations described with respect to routine 300, which can be specially programmed to cause vessel physiology modeling system 100 to perform the operations described with reference to routine 300 and FIG. 3. As another example, computer executable instructions 602 can include instructions 120, ML model 514, and/or training algorithm 516. Examples of computer-readable storage medium 600 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 602 may include any suitable type of code, such as source code, compiled code, interpreted code, executable code, static code, dynamic code, object-oriented code, visual code, and the like.

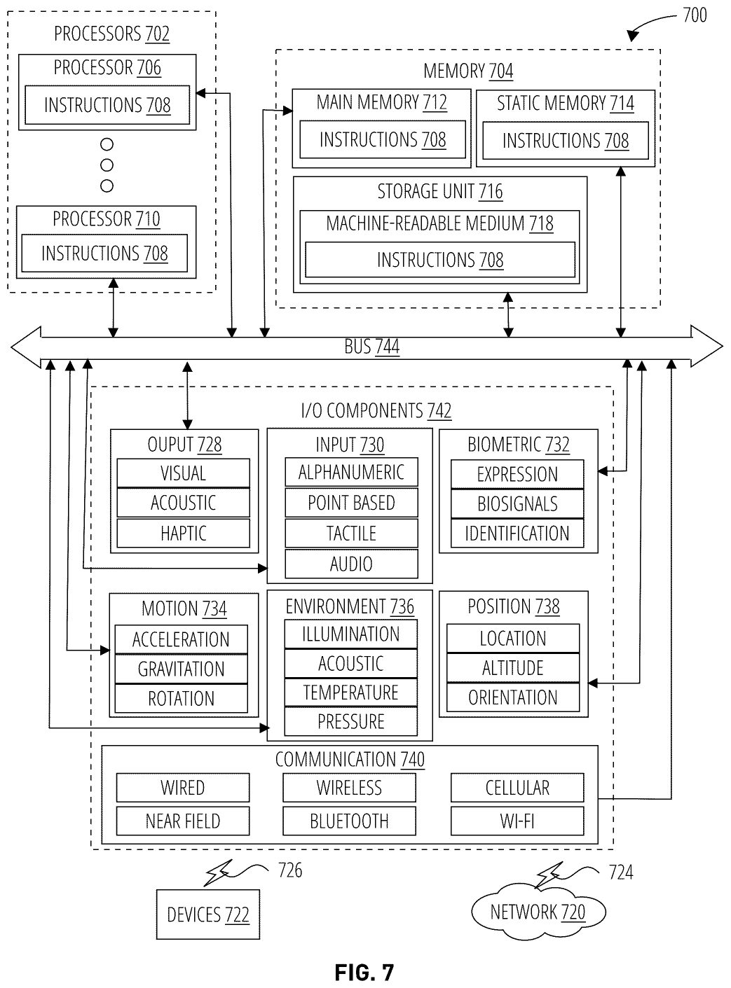

FIG. 7 illustrates a diagrammatic representation of a machine 700 in the form of a computer system within which a set of instructions may be executed for causing the machine to perform any one or more of the methodologies discussed herein. More specifically, FIG. 7 shows a diagrammatic representation of the machine 700 in the example form of a computer system, within which instructions 708 (e.g., software, a program, an application, an applet, an app, or other executable code) for causing the machine 700 to perform any one or more of the methodologies discussed herein may be executed. For example, the instructions 708 may cause the machine 700 to execute instructions 120, routine 300 of FIG. 3, training algorithm 516, or the like. More generally, the instructions 708 may cause the machine 700 to generate a 3D model or a vessel physiology from a single angiogram, a series of IVUS images, and vessel pressure measurements as described herein.

The instructions 708 transform the general, non-programmed machine 700 into a particular machine 700 programmed to carry out the described and illustrated functions in a specific manner. In alternative embodiments, the machine 700 operates as a standalone device or may be coupled (e.g., networked) to other machines. In a networked deployment, the machine 700 may operate in the capacity of a server machine or a client machine in a server-client network environment, or as a peer machine in a peer-to-peer (or distributed) network environment. The machine 700 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 708, sequentially or otherwise, that specify actions to be taken by the machine 700. Further, while only a single machine 700 is illustrated, the term “machine” shall also be taken to include a collection of machines 200 that individually or jointly execute the instructions 708 to perform any one or more of the methodologies discussed herein.

The machine 700 may include processors 702, memory 704, and I/O components 742, which may be configured to communicate with each other such as via a bus 744. In an example embodiment, the processors 702 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 706 and a processor 710 that may execute the instructions 708. The term “processor” is intended to include multi-core processors that may comprise two or more independent processors (sometimes referred to as “cores”) that may execute instructions contemporaneously. Although FIG. 7 shows multiple processors 702, the machine 700 may include a single processor with a single core, a single processor with multiple cores (e.g., a multi-core processor), multiple processors with a single core, multiple processors with multiples cores, or any combination thereof.

The memory 704 may include a main memory 712, a static memory 714, and a storage unit 716, both accessible to the processors 702 such as via the bus 744. The main memory 704, the static memory 714, and storage unit 716 store the instructions 708 embodying any one or more of the methodologies or functions described herein. The instructions 708 may also reside, completely or partially, within the main memory 712, within the static memory 714, within machine-readable medium 718 within the storage unit 716, within at least one of the processors 702 (e.g., within the processor's cache memory), or any suitable combination thereof, during execution thereof by the machine 700.

The I/O components 742 may include a wide variety of components to receive input, provide output, produce output, transmit information, exchange information, capture measurements, and so on. The specific I/O components 742 that are included in a particular machine will depend on the type of machine. For example, portable machines such as mobile phones will likely include a touch input device or other such input mechanisms, while a headless server machine will likely not include such a touch input device. It will be appreciated that the I/O components 742 may include many other components that are not shown in FIG. 7. The I/O components 742 are grouped according to functionality merely for simplifying the following discussion and the grouping is in no way limiting. In various example embodiments, the I/O components 742 may include output components 728 and input components 730. The output components 728 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 730 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 742 may include biometric components 732, motion components 734, environmental components 736, or position components 738, among a wide array of other components. For example, the biometric components 732 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 734 may include acceleration sensor components (e.g., accelerometer), gravitation sensor components, rotation sensor components (e.g., gyroscope), and so forth. The environmental components 736 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 738 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 742 may include communication components 740 operable to couple the machine 700 to a network 720 or devices 722 via a coupling 724 and a coupling 726, respectively. For example, the communication components 740 may include a network interface component or another suitable device to interface with the network 720. In further examples, the communication components 740 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 722 may be another machine or any of a wide variety of peripheral devices (e.g., a peripheral device coupled via a USB).

Moreover, the communication components 740 may detect identifiers or include components operable to detect identifiers. For example, the communication components 740 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 740, such as location via Internet Protocol (IP) geolocation, location via Wi-Fi® signal triangulation, location via detecting an NFC beacon signal that may indicate a particular location, and so forth.

The various memories (i.e., memory 704, main memory 712, static memory 714, and/or memory of the processors 702) and/or storage unit 716 may store one or more sets of instructions and data structures (e.g., software) embodying or utilized by any one or more of the methodologies or functions described herein. These instructions (e.g., the instructions 708), when executed by processors 702, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 720 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 720 or a portion of the network 720 may include a wireless or cellular network, and the coupling 724 may be a Code Division Multiple Access (CDMA) connection, a Global System for Mobile communications (GSM) connection, or another type of cellular or wireless coupling. In this example, the coupling 724 may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 708 may be transmitted or received over the network 720 using a transmission medium via a network interface device (e.g., a network interface component included in the communication components 740) and utilizing any one of several well-known transfer protocols (e.g., hypertext transfer protocol (HTTP)). Similarly, the instructions 708 may be transmitted or received using a transmission medium via the coupling 726 (e.g., a peer-to-peer coupling) to the devices 722. The terms “transmission medium” and “signal medium” mean the same thing and may be used interchangeably in this disclosure. The terms “transmission medium” and “signal medium” shall be taken to include any intangible medium that can store, encoding, or carrying the instructions 708 for execution by the machine 700, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. An apparatus for a vascular imaging medical device, comprising:
a processor arranged to be coupled to an intravascular imaging device and a fluoroscope device; and
a memory device coupled to the processor, the memory device comprising instructions, which when executed by the processor cause the apparatus to:
receive, from the fluoroscope device, an angiographic image if a vessel of a patient;
receive, from the intravascular imaging device, a plurality of images associated with the vessel of the patient, the plurality of images comprising multidimensional and multivariate images; and
generate a three-dimensional (3D) model of a physiology of the vessel from the angiographic image and the plurality of images.

2. The apparatus of claim 1, the instructions when executed by the processor further cause the apparatus to:
generate a graphical information element comprising an indication of the 3D model; and
cause the graphical information element to be displayed on a display coupled to the computing device.

3. The apparatus of claim 1, the instructions when executed by the processor further cause the apparatus to co-register the angiographic image and the plurality of images.

4. The apparatus of claim 3, the instructions when executed by the processor further cause the apparatus to:
identify a start point of a pull-back operation associated with the plurality of images on the vessel represented in the angiographic image;
identify an end point of the pull-back operation associated with the plurality of images on the vessel represented in the angiographic image; and
identify a centerline of the vessel between start point and the end point.

5. The apparatus of claim 4, the instructions when executed by the processor further cause the apparatus to:
identify a plurality of side branches of the vessel on the angiographic image and in the plurality of images; and
match a one of the plurality of side branches identified on the angiographic image with a one of the plurality of side branches identified in the plurality of images.

6. The apparatus of claim 5, the instructions when executed by the processor further cause the apparatus to map frames of the plurality of images with locations along the centerline of the vessel on the angiographic image.

7. The apparatus of claims 1, the instructions when executed by the processor further cause the apparatus to generate assessments of the vessel, wherein the assessments comprise a diameter of the vessel, an area of the vessel, or a diameter and area of the vessel and wherein the assessments comprise a diameter of the lumen, an area of the lumen, or a diameter and area of the lumen.

8. The apparatus of claims 1, the instructions when executed by the processor further cause the apparatus to:
receive an indication of an additional physiological characteristic of the vessel of the patient; and
generate the 3D model of the physiology of the vessel from the angiographic image, the plurality of images, and the additional physiological characteristic of the vessel,
wherein the additional physiological characteristic of the vessel comprises pressure or flow.

9. The apparatus of claim 1, the instructions when executed by the processor further cause the apparatus to generate an inference of the 3D model of the physiologic of the vessel from a machine learning (ML) model based in part on applying the angiographic image and the plurality of images as inputs to the ML model.

10. The apparatus of claim 9, wherein the ML model is trained based in part on a supervised learning training algorithm with expected outputs of the ML model derived based on a computation fluid dynamics (CFD) model, wherein the CFD model takes an angiographic image and a plurality of images as input and generates a 3D vessel physiology model as output.

11. A computer-readable storage device, comprising instructions executable by a processor of a computing device coupled to an intravascular imaging device and a fluoroscope device, wherein when executed, the instructions cause the computing device to:
receive, from the fluoroscope device, an angiographic image if a vessel of a patient;
receive, from the intravascular imaging device, a plurality of images associated with the vessel of the patient, the plurality of images comprising multidimensional and multivariate images; and
generate a three-dimensional (3D) model of a physiology of the vessel from the angiographic image and the plurality of images.

12. The computer-readable storage device of claim 11, the instructions when executed by the processor further cause the computing device to:
generate a graphical information element comprising an indication of the 3D model; and
cause the graphical information element to be displayed on a display coupled to the computing device.

13. The computer-readable storage device of claim 11, the instructions when executed by the processor further cause the computing device to:
identify a start point of a pull-back operation associated with the plurality of images on the vessel represented in the angiographic image;
identify an end point of the pull-back operation associated with the plurality of images on the vessel represented in the angiographic image;
identify a centerline of the vessel between start point and the end point;
identify a plurality of side branches of the vessel on the angiographic image and in the plurality of images;
match a one of the plurality of side branches identified on the angiographic image with a one of the plurality of side branches identified in the plurality of images; and
map frames of the plurality of images with locations along the centerline of the vessel on the angiographic image.

14. The computer-readable storage device of claim 11, the instructions when executed by the processor further cause the computing device to generate assessments of the vessel, wherein the assessments comprise a diameter of the vessel, an area of the vessel, or a diameter and area of the vessel and wherein the assessments comprise a diameter of the lumen, an area of the lumen, or a diameter and area of the lumen.

15. The computer-readable storage device of claim 11, the instructions when executed by the processor further cause the computing device to:
receive an indication of an additional physiological characteristic of the vessel of the patient; and
generate the 3D model of the physiology of the vessel from the angiographic image, the plurality of images, and the additional physiological characteristic of the vessel,
wherein the additional physiological characteristic of the vessel comprises pressure or flow.

16. The computer-readable storage device of claim 11, the instructions when executed by the processor further cause the computing device to generate an inference of the 3D model of the physiologic of the vessel from a machine learning (ML) model based in part on applying the angiographic image and the plurality of images as inputs to the ML model, wherein the ML model is trained based in part on a supervised learning training algorithm with expected outputs of the ML model derived based on a computation fluid dynamics (CFD) model, wherein the CFD model takes an angiographic image and a plurality of images as input and generates a 3D vessel physiology model as output.

17. A computer-implemented method for a vascular imaging medical device, comprising:
receiving, at a computer from a fluoroscope device, an angiographic image if a vessel of a patient;
receiving, at the computer from an intravascular imaging device, a plurality of images associated with the vessel of the patient, the plurality of images comprising multidimensional and multivariate images; and
generating a three-dimensional (3D) model of a physiology of the vessel from the angiographic image and the plurality of images.

18. The computer-implemented method of claim 17, comprising:
generating a graphical information element comprising an indication of the 3D model; and
causing the graphical information element to be displayed on a display coupled to the computing device.

19. The computer-implemented method of claim 17, comprising:
identifying a start point of a pull-back operation associated with the plurality of images on the vessel represented in the angiographic image;
identifying an end point of the pull-back operation associated with the plurality of images on the vessel represented in the angiographic image;
identifying a centerline of the vessel between start point and the end point;
identifying a plurality of side branches of the vessel on the angiographic image and in the plurality of images;
matching a one of the plurality of side branches identified on the angiographic image with a one of the plurality of side branches identified in the plurality of images; and
mapping frames of the plurality of images with locations along the centerline of the vessel on the angiographic image.

20. The computer-implemented method of claim 17, comprising:
receiving an indication of an additional physiological characteristic of the vessel of the patient; and
generating the 3D model of the physiology of the vessel from the angiographic image, the plurality of images, and the additional physiological characteristic of the vessel,
wherein the additional physiological characteristic of the vessel comprises pressure or flow.
